(19) United States
(12) Patent Application Publication
(10) Pub. No.: US 2025/0157809 A1
(43) Pub. Date: May 15, 2025

(54) THIN-FILM FORMING COMPOSITION INCLUDING ALUMINUM COMPOUND, METHOD OF FORMING THIN FILM BY USING THE THIN-FILM FORMING COMPOSITION, AND METHOD OF MANUFACTURING SEMICONDUCTOR DEVICE

(71) Applicant: Samsung Electronics Co., Ltd., Suwon-si (KR)

(72) Inventors: Daeun Kim, Suwon-si (KR); Seung-Min Ryu, Suwon-si (KR); Gyu-Hee Park, Suwon-si (KR); Joongjin Park, Daejeon-si (KR); Sangick Lee, Daejeon-si (KR); Sanghun Lee, Daejeon-si (KR); Seunghyeon Lee, Daejeon-si (KR); Sejin Jang, Daejeon-si (KR); Youn Joung Cho, Suwon-si (KR)

(73) Assignees: Samsung Electronics Co., Ltd., Suwon-si (KR); DNF Co., Ltd., Daejeon (KR)

(21) Appl. No.: 18/934,625

(22) Filed: Nov. 1, 2024

(30) Foreign Application Priority Data
Nov. 14, 2023 (KR) 10-2023-0157689

(51) Int. Cl. H01L 21/02; C07C 319/20; C23C 16/40; C23C 16/44; C23C 16/455

(52) U.S. Cl. CPC H01L 21/02205; C07C 319/20; C23C 16/403; C23C 16/4408; C23C 16/45553; H01L 21/02178; H01L 21/0228

(57) ABSTRACT

A thin-film forming composition includes an aluminum compound represented by General Formula (1).

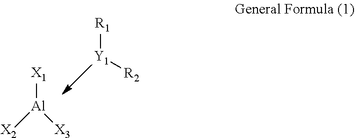

In General Formula (1), X₁, X₂, and X₃ are each independently a halogen atom, R₁ and R₂ are each independently a C1-C5 alkyl group, and Y₁ is a chalcogen atom.

### CROSS-REFERENCE TO RELATED APPLICATION

This application is based on and claims priority under 35 U.S.C. § 119 to Korean Patent Application No. 10-2023-0157689, filed on Nov. 14, 2023, in the Korean Intellectual Property Office, the disclosure of which is incorporated by reference herein in its entirety.

### BACKGROUND

Aspects of the inventive concept relate to a thin-film forming composition including an aluminum compound, a method of forming a thin film by using the thin-film forming composition, and a method of manufacturing a semiconductor device.

Due to the advance in electronics technology, semiconductor devices have been rapidly down-scaled in recent years, and thus, patterns constituting semiconductor devices have been increasingly fine-sized. Therefore, there is a need to develop a thin-film forming raw compound, which allows a thin film having a relatively uniform thickness to be formed by securing thermal stability in forming an aluminum-containing thin film.

### SUMMARY

Aspects of the inventive concept provide a thin-film forming composition including an aluminum compound as a raw compound for forming an aluminum-containing thin film, the aluminum compound allowing a relatively uniform-thickness thin film to be obtained in forming an aluminum-containing thin film and being capable of providing excellent thermal stability, process stability, and mass productivity.

Aspects of the inventive concept also provide a method of forming an aluminum-containing thin film, the method allowing a uniform-thickness thin film to be obtained in forming an aluminum-containing thin film and allowing excellent thermal stability, process stability, and mass productivity to be provided, and aspects of the inventive concept also provide a method of manufacturing a semiconductor device, which may provide excellent electrical characteristics, by using the method of forming an aluminum-containing thin film.

According to an aspect of the inventive concept, a thin-film forming composition includes an aluminum compound represented by General Formula (1),

### General Formula (1)

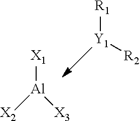

wherein, in General Formula (1), X₁, X₂, and X₃ are each independently a halogen atom, R₁ and R₂ are each independently a C1-C5 alkyl group, and Y₁ is a chalcogen atom.

According to another aspect of the inventive concept, a method of forming a thin film includes forming an aluminum-containing film on a substrate by using a thin-film forming composition including an aluminum compound represented by General Formula (1).

According to another aspect of the inventive concept, a method of manufacturing a semiconductor device includes forming a lower structure on a substrate, and forming an aluminum-containing film on the lower structure by using a thin-film forming composition that includes an aluminum compound represented by General Formula (1).

### BRIEF DESCRIPTION OF THE DRAWINGS

Embodiments will be more clearly understood from the following detailed description taken in conjunction with the accompanying drawings in which:

FIG. 1 is a flowchart illustrating a method of forming a thin film, according to some embodiments;

FIG. 2 is a flowchart specifically illustrating a method of forming an aluminum-containing film, according to some embodiments;

FIGS. 3A to 3J are cross-sectional views illustrating a method of manufacturing a semiconductor device, according to some embodiments;

FIG. 4 is a graph illustrating a result of differential scanning calorimetry (DSC) analysis of an aluminum compound according to an embodiment;

FIG. 5 is a graph illustrating a result of thermogravimetric analysis (TGA) of an aluminum compound according to an embodiment;

FIG. 6 is a graph illustrating a result of measuring a vapor pressure of an aluminum compound according to an embodiment depending on a change in temperature;

FIG. 7 is a graph illustrating a result of evaluating a deposition rate of an aluminum oxide film, which is formed by using an aluminum compound according to an embodiment as a precursor, depending on a substrate temperature;

FIG. 8 is a graph illustrating a result of X-ray photoelectron spectroscopy (XPS) depth profiling analysis of an aluminum oxide film, which is formed by using an aluminum compound according to an embodiment as a precursor, for analyzing the concentration composition of the aluminum oxide film;

FIG. 9 is a transmission electron microscopy (TEM) image of an aluminum oxide film, which is formed by using an aluminum compound according to an embodiment as a precursor, for analyzing step coverage properties of the aluminum compound;

FIG. 10 is a graph illustrating a result of evaluating a deposition rate of an aluminum oxide film, which is formed by using an aluminum compound according to a comparison example, depending on a substrate temperature; and

FIG. 11 is a graph illustrating respective results of deposition rates of an aluminum compound according to an embodiment and an aluminum compound according to a comparison example, depending on a substrate temperature.

### DETAILED DESCRIPTION OF THE EMBODIMENTS

Hereinafter, embodiments of the inventive concept will be described in detail with reference to the accompanying drawings. Like components are denoted by like reference numerals throughout the specification, and repeated descriptions thereof are omitted.

An aluminum compound, which is included in a thin-film forming composition, according to some embodiments may be represented by General Formula (1).

### General Formula (1)

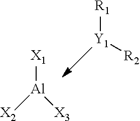

In General Formula (1), X₁, X₂, and X₃ are each independently a halogen atom, R₁ and R₂ are each independently a C1-C5 alkyl group, and Y₁ is a chalcogen atom. For example each of X₁, X₂, and X₃ can be the same type of halogen atom, all three of X₁, X₂, and X₃ can be different types of halogen atoms, or two of X₁, X₂, and X₃ can be the same type of halogen atom and the other of X₁, X₂, and X₃ can be a different type of halogen atom. Similarly, each of R₁ and R₂ may be the same type of alkyl group, or may be a different type of alkyl group.

Each alkyl group may be a linear, branched, or cyclic alkyl group. Examples of the linear alkyl group include, but are not limited to, a methyl group, an ethyl group, a propyl group, a butyl group, and the like. Examples of the branched alkyl group may include, but are not limited to, an i-propyl group, a neo-butyl group, an i-butyl group, and a t-butyl group. Examples of the cyclic alkyl group may include, but are not limited to, a cyclopropyl group, a cyclopentyl group, and the like.

In some embodiments, R₁ and R₂ may each be independently one of a C1-C3 alkyl group.

In some embodiments, X₁, X₂, and X₃ may each be one selected independently from the group consisting of Cl, Br, and I.

In some embodiments, Y₁ may be one selected from the group consisting of O, S, and Se.

In some embodiments, the aluminum compound of the thin-film forming composition may be represented by General Formula (2).

### General Formula (2)

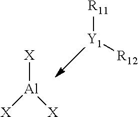

In General Formula (2), X is a halogen atom, R₁₁ and R₁₂ are each independently a C1-C5 alkyl group, and Y₁ is a chalcogen atom.

In some embodiments, the aluminum compound of the thin-film forming composition may have a structure of one selected from the following chemical formulae.

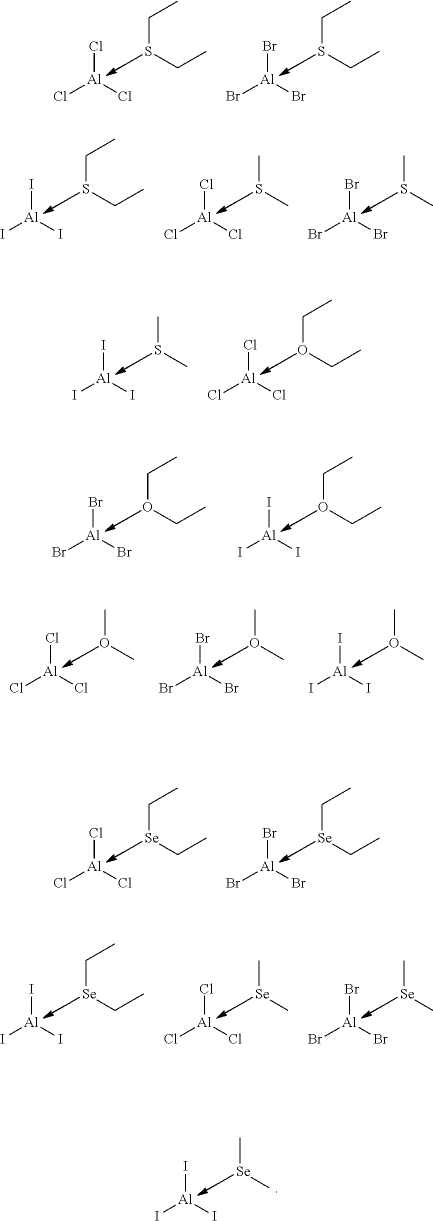

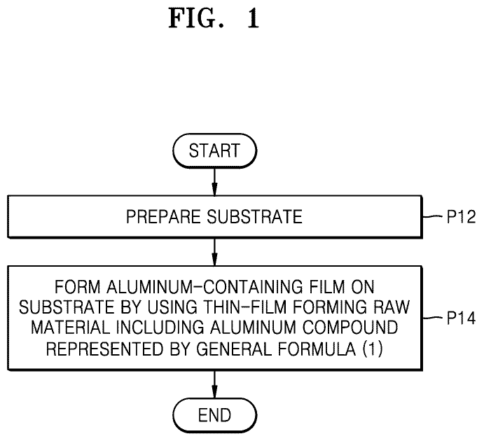

FIG. 1 is a flowchart illustrating a method of forming a thin film, according to some embodiments.

Referring to FIG. 1, first, in process P12, a substrate may be provided into a reaction chamber. Next, the provided substrate may be heated such that the substrate is maintained at about 200° C. to about 500° C. Next, in process P14, an aluminum-containing film may be formed on the substrate by using a thin-film forming composition including an aluminum compound represented by General Formula (1). In some embodiments, the aluminum compound of the thin-film forming composition used in process P14 may be liquid at room temperature. In some embodiments, the aluminum compound of the thin-film forming composition may be represented by General Formula (2).

In some embodiments, the thin-film forming composition may include at least one aluminum compound from among aluminum compounds according to some embodiments and may not include other metal compounds and semimetal compounds. In some embodiments, the thin-film forming composition may include an intended metal-containing or semimetal-containing compound (hereinafter, referred to as “another precursor or other precursors”) in addition to the aluminum compound. In some embodiments, the thin-film forming composition may include an organic solvent or a nucleophilic reagent in addition to the aluminum compound.

Examples of the other precursors, which may be used for a thin-film forming composition in the method of forming a thin film (which may also be referred to as the thin-film forming method) according to some embodiments, may include at least one Si or metal compound selected from compounds including, as ligands, hydrides, hydroxides, halides, azides, alkyl groups, alkenyl groups, cycloalkyl groups, allyl groups, alkynyl groups, amino groups, dialkylaminoalkyl groups, monoalkylamino groups, dialkylamino groups, diamino groups, di(silyl-alkyl)amino groups, di(alkyl-silyl)amino groups, disilylamino groups, alkoxy groups, alkoxyalkyl groups, hydrazides, phosphides, nitriles, dialkylaminoalkoxy groups, alkoxyalkyldialkylamino groups, siloxy groups, diketonates, cyclopentadienyl groups, silyl groups, pyrazolates, guanidinates, phosphoguanidinates, amidinates, phosphoamidinates, ketoiminates, diketoiminates, and carbonyl groups.

A metal in the other precursors, which may be used for a thin-film forming composition in the thin-film forming method according to some embodiments, may include Ti, Ta, Mg, Ca, Sr, Ba, Ra, Sc, Y, Zr, Hf, V, Nb, Cr, Mo, W, Mn, Fe, Os, Co, Rh, Ir, Ni, Pd, Pt, Cu, Ag, Au, Zn, Cd, Ga, In, Ge, Sn, Pb, Sb, Bi, La, Ce, Pr, Nd, Pm, Sm, Eu, Gd, Tb, Dy, Ho, Er, Tm, Yb, or the like, but the inventive concept is not limited to the examples of metals set forth above.

The thin-film forming composition including the aluminum compound according to some embodiments may be suitably used for an atomic layer deposition (ALD) process, and the aluminum compound of the thin-film forming composition according to some embodiments may be used as an Al precursor for an ALD process in a thin-film forming process required to manufacture a semiconductor device.

In the thin-film forming method according to some embodiments, an aluminum-containing film may be formed in a reaction chamber of a deposition apparatus by using the thin-film forming composition including the aluminum compound having a structure of General Formula (1). In some embodiments, to form the aluminum-containing film, the thin-film forming composition including only the aluminum compound having a structure of General Formula (1) may be supplied onto the substrate. In some embodiments, to form the aluminum-containing film, the thin-film forming composition, which includes a mixture of the aluminum compound and at least one of an organic solvent, a reactive gas, and a precursor compound including a different metal from aluminum, may be supplied onto the substrate.

When an aluminum nitride film is formed by the thin-film forming method according to some embodiments, the reactive gas may be selected from N₂, NH₃, monoalkylamines, dialkylamines, trialkylamines, organic amine compounds, hydrazine compounds, and combinations thereof.

When an aluminum oxide film is formed by the thin-film forming method according to some embodiments, the reactive gas may include an oxidizing gas selected from O₂, O₃, plasma O₂, H₂O, NO₂, NO, N₂O (nitrous oxide), CO₂, H₂O₂, HCOOH, CH₃COOH, (CH₃CO)₂O, and combinations thereof.

In some embodiments, the reactive gas may include a reducing gas, for example, H₂.

In some embodiments, the reactive gas may be selected from inert gases, for example, Ar, He, and a combination thereof.

The aluminum compound and the reactive gas may be sequentially or simultaneously supplied onto the substrate.

In the thin-film forming method according to some embodiments, the substrate for forming a thin film may be or include a silicon substrate; a ceramic substrate, such as SiN, TiN, TaN, TiO, TiN, RuO, ZrO, HfO, or LaO; a metal substrate, such as ruthenium; or the like.

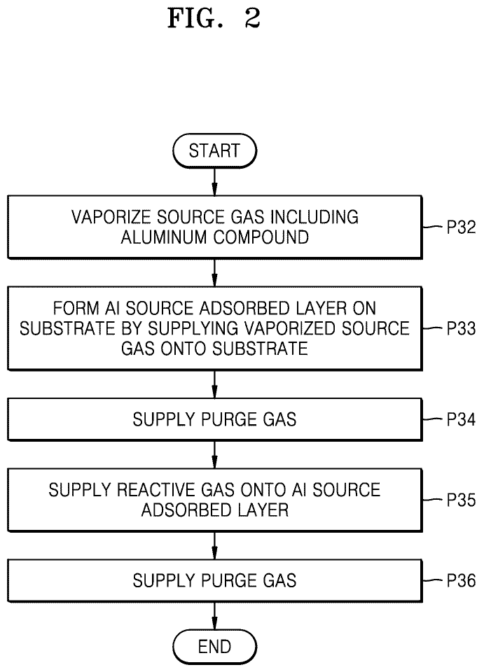

FIG. 2 is a flowchart specifically illustrating a method of forming an aluminum-containing film, according to some embodiments. The method of forming an aluminum-containing film by an ALD process according to process P14 of FIG. 1 is described in detail with reference to FIG. 2.

Referring to FIG. 2, in process P32, a source gas including an aluminum compound is vaporized. The aluminum compound may include an aluminum compound having a structure represented by General Formula (1).

In process P33, while the substrate provided into the reaction chamber is maintained at a temperature of about 200° C. to about 500° C., the source gas vaporized according to process P32 is supplied onto the substrate by using a carrier gas, thereby forming an Al source-adsorbed layer on the substrate. The carrier gas may include or be, for example, an inert gas, such as Ar, He, or Ne, N₂ gas, or the like. The adsorbed layer including a chemisorbed layer and a physisorbed layer of the vaporized source gas may be formed on the substrate by supplying the vaporized source gas onto the substrate.

In process P34, while the substrate provided into the reaction chamber is maintained at a temperature of about 200° C. to about 500° C., a purge gas is supplied onto the substrate, thereby removing unnecessary byproducts on the substrate. The purge gas may include or be, for example, an inert gas, such as Ar, He, or Ne, N₂ gas, or the like.

In process P35, while the substrate provided into the reaction chamber is maintained at a temperature of about 200° C. to about 500° C., a reactive gas may be supplied onto the Al source-adsorbed layer formed on the substrate.

When an aluminum nitride film is formed as the aluminum-containing film, the reactive gas may be selected from NH₃, monoalkylamines, dialkylamines, trialkylamines, organic amine compounds, hydrazine compounds, and combinations thereof. When an aluminum oxide film is formed as the aluminum-containing film, the reactive gas may include an oxidizing gas selected from O₂, O₃, plasma O₂, H₂O, NO₂, NO, N₂O, CO₂, H₂O₂, HCOOH, CH₃COOH, (CH₃CO)₂O, and combinations thereof. In some embodiments, the reactive gas may include a reducing gas, for example, H₂. In some embodiments, the reactive gas may be selected from inert gases, for example, Ar, He, and a combination thereof.

In process P36, unnecessary byproducts on the substrate may be removed by supplying a purge gas onto the substrate.

To form the aluminum-containing film on the substrate by the thin-film forming method according to some embodiments, the aluminum compound having a structure represented by General Formula (1) and at least one of another precursor, a reactive gas, a carrier gas, and a purge gas may be simultaneously or sequentially supplied onto the substrate. In some embodiments, the aluminum compound may be represented by General Formula (2) described above, and here, X is a halogen atom, R₁₁ and R₁₂ are each independently a C1-C5 alkyl group, and Y₁ is a chalcogen atom.

When an aluminum-containing film is formed by an ALD process according to some embodiments, to control the aluminum-containing film with an intended thickness, the number of ALD cycles may be adjusted.

For example, when an aluminum-containing film is formed by an ALD process, energy, such as plasma, light, or voltage, may be applied. A time point of applying the energy as such may be variously selected. For example, at a time point when the source gas including the aluminum compound is introduced into the reaction chamber, at a time point when the source gas is adsorbed onto the substrate, at a time point of an exhaust process by the purge gas, at a time point when the reactive gas is introduced into the reaction chamber, or between each of these time points, the energy such as plasma, light, or voltage may be applied.

According to the thin-film forming method according to some embodiments, by appropriately selecting an aluminum compound according to some embodiments, another precursor used together with the aluminum compound, a reactive gas, and process conditions for thin-film formation, various aluminum-containing films may be formed.

In some embodiments, the aluminum-containing film formed by the thin-film forming method according to some embodiments may include an aluminum oxide film represented by Al₂O₃, an aluminum nitride film represented by AlN, an aluminum alloy film, a composite oxide film including an aluminum alloy, or the like. The composite oxide film may include a composite oxide film of Ti and Al, a composite oxide film of Ta and Al, or the like, but the inventive concept is not limited to the examples set forth above.

The aluminum-containing film formed by the thin-film forming method according to some embodiments may be used for various applications. For example, the aluminum-containing film may be used for a tunnel barrier of a gate dielectric film in a 3-dimensional charge trap flash (CTF) cell, a gate of a transistor, a conductive barrier film in a metal wiring line such as a copper wiring line, a dielectric film of a capacitor, an electrode of a resistance-variable memory device, a barrier metal film for liquid crystals, a member for thin-film solar cells, a member for semiconductor facilities, a nano-structure, or the like, but the applications of the aluminum-containing film are not limited to the examples set forth above.

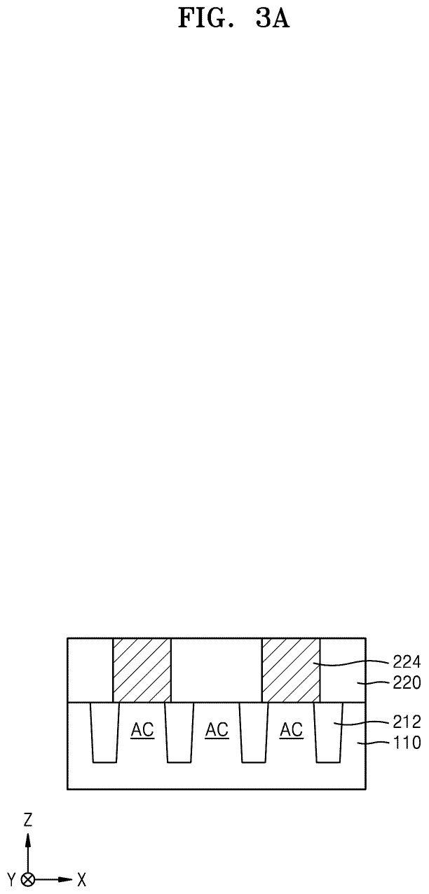

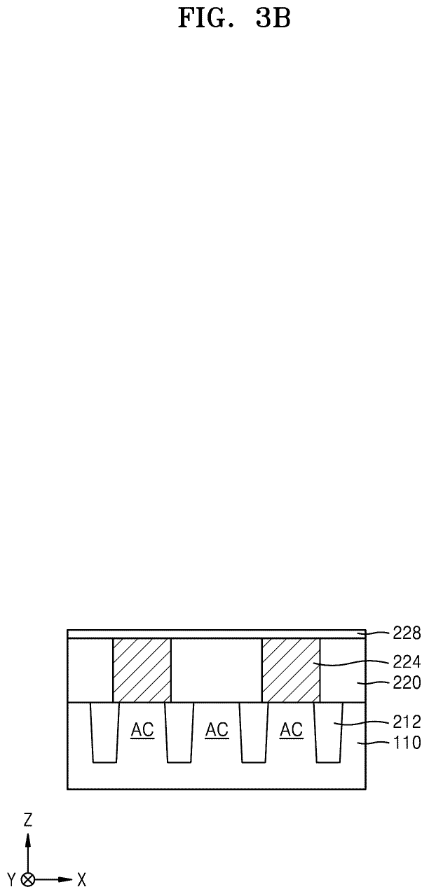

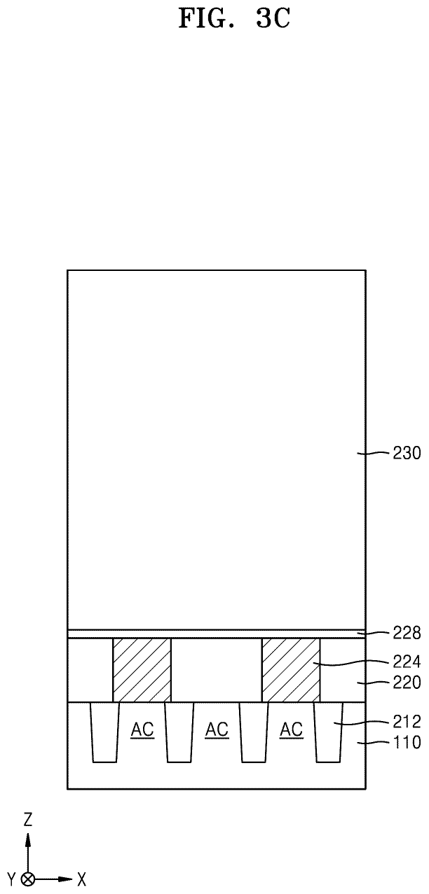

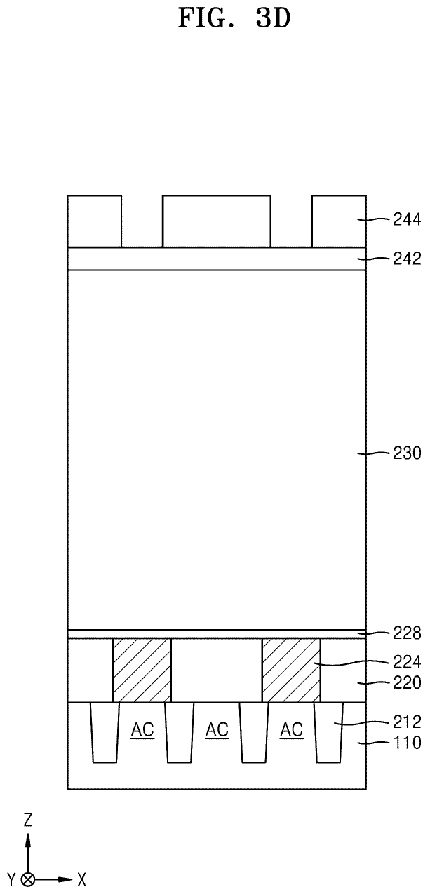

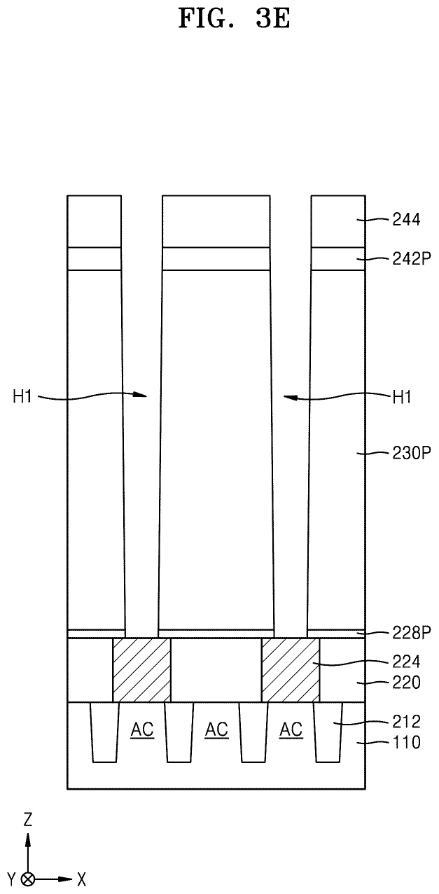

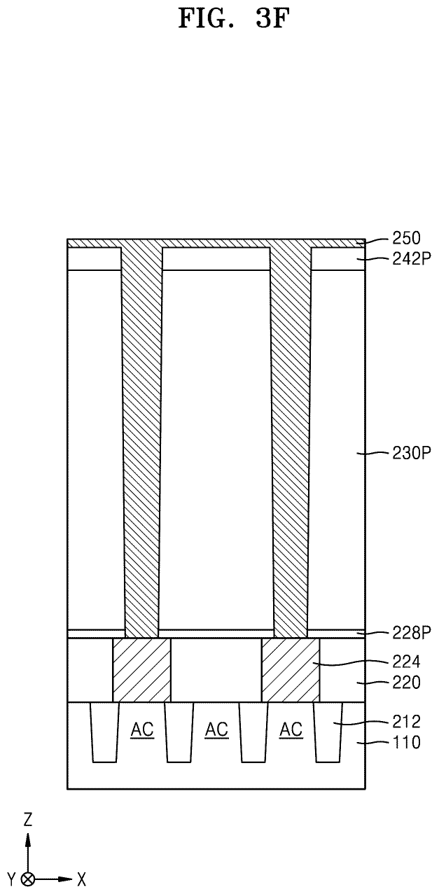

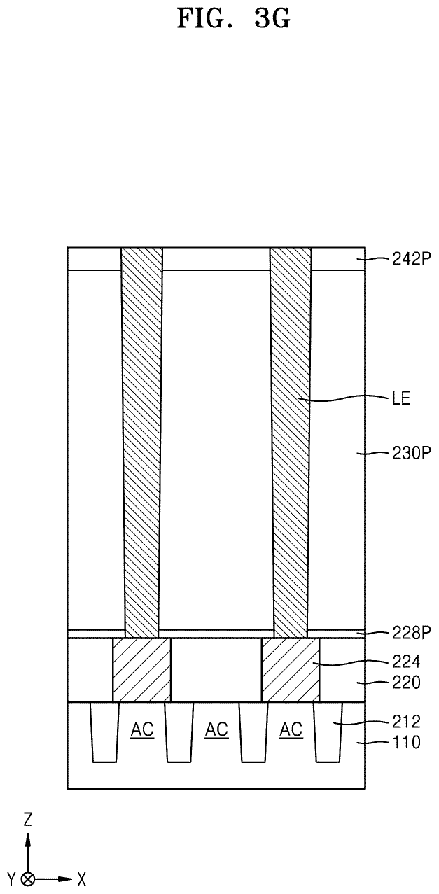

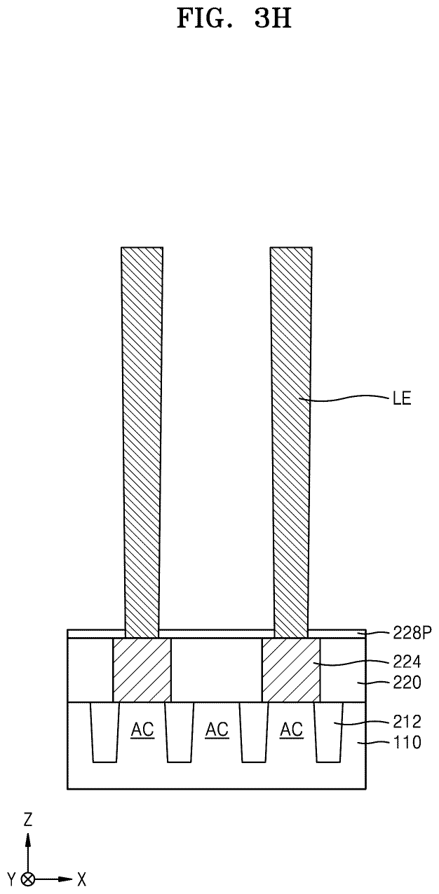

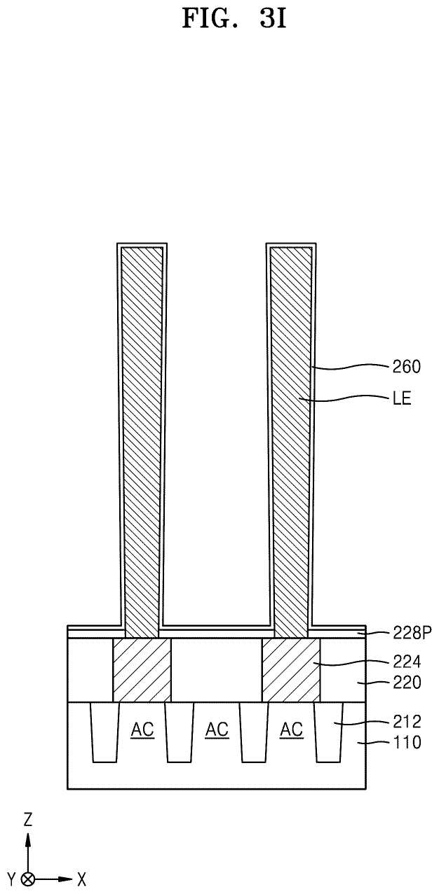

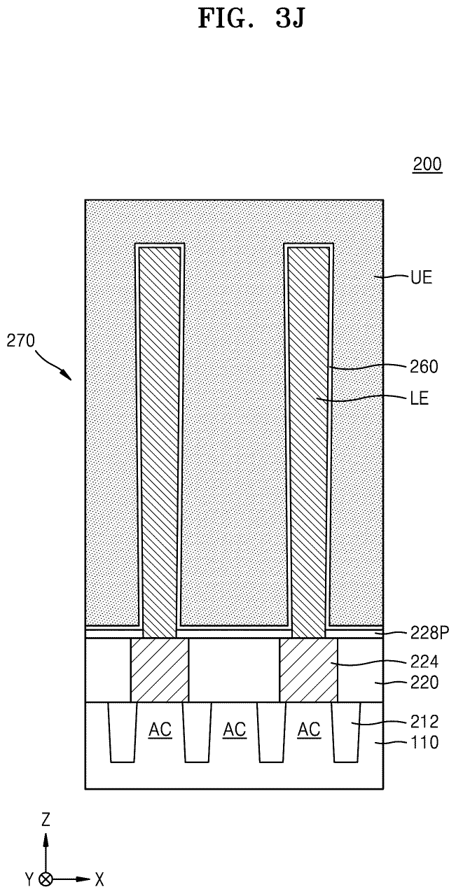

FIGS. 3A to 3J are cross-sectional views illustrating a method of manufacturing a semiconductor device 200 (see FIG. 3J), according to some embodiments. The semiconductor device 200 may be, for example, a semiconductor memory chip.

Referring to FIG. 3A, an interlayer dielectric 220 (e.g., interlayer dielectric layer) may be formed on a substrate 110 including a plurality of active regions AC, and then, a plurality of conductive regions 224 (e.g., conductive lines) may be formed through the interlayer dielectric 220 to be connected to the plurality of active regions AC.

The plurality of active regions AC may be defined by a plurality of device isolation films 212. The interlayer dielectric 220 may include or be a silicon oxide film. The plurality of conductive regions 224 may each include or be polysilicon, a metal, a conductive metal nitride, a metal silicide, or a combination thereof.

Referring to FIG. 3B, an insulating layer 228 may be formed to cover the interlayer dielectric 220 and the plurality of conductive regions 224.

The insulating layer 228 may be used as an etch-stop layer. The insulating layer 228 may include or be formed of an insulating material having etch selectivity with respect to the interlayer dielectric 220 and to a mold film 230 (see FIG. 3C) formed in a subsequent process. In some embodiments, the insulating layer 228 may include or be formed of silicon nitride, silicon oxynitride, or a combination thereof.

Referring to FIG. 3C, the mold film 230 may be formed on the insulating layer 228. The mold film 230 may include or be an oxide film. In some embodiments, the mold film 230 may include or be a support film (not shown). The support film may include or be formed of a material having etch selectivity with respect to the mold film 230.

Referring to FIG. 3D, a sacrificial film 242 and a mask pattern 244 may be formed in the stated order on the mold film 230.

The sacrificial film 242 may include or be an oxide film. The sacrificial film 242 may protect the support film of the mold film 230. The mask pattern 244 may include or be an oxide film, a nitride film, a polysilicon film, a photoresist film, or a combination thereof. A region in which a lower electrode of a capacitor is to be formed may be defined by the mask pattern 244.

Referring to FIG. 3E, the sacrificial film 242 and the mold film 230 may be etched by using the mask pattern 244 as an etch mask and using the insulating layer 228 as an etch-stop layer, thereby forming a sacrificial pattern 242P and a mold pattern 230P, which define a plurality of holes H1. In the etching process set forth above, the insulating layer 228 may also be etched due to over-etching, and thus, an insulating pattern 228P may be formed to expose the plurality of conductive regions 224.

Referring to FIG. 3F, the mask pattern 244 (see FIG. 3E) may be removed, and then, a lower electrode forming conductive film 250 may be formed to cover respective exposed surfaces of the sacrificial film 242 and the plurality of holes H1 (see FIG. 3E).

The lower electrode forming conductive film 250 may include or be formed of a doped semiconductor, a conductive metal nitride, a metal, a metal silicide, a conductive oxide, or a combination thereof. For example, the lower electrode forming conductive film 250 may include or be formed of TiN, TiAlN, TaN, TaAlN, W, WN, Ru, RuO₂, SrRuO₃, Ir, IrO₂, Pt, PtO, (Ba,Sr)RuO₃ (BSRO), CaRuO₃ (CRO), (La,Sr)CoO₃ (LSCO), or a combination thereof. To form the lower electrode forming conductive film 250, a chemical vapor deposition (CVD) process, a metal organic CVD (MOCVD) process, or an ALD process may be used.

Referring to FIG. 3G, an upper portion of the lower electrode forming conductive film 250 may be removed, thereby separating the lower electrode forming conductive film 250 into a plurality of lower electrodes LE.

To form the plurality of lower electrodes LE, an upper portion of the lower electrode forming conductive film 250 and the sacrificial pattern 242P (see FIG. 3F) may be removed by an etch-back process or a chemical mechanical polishing (CMP) process such that the upper surface of the mold pattern 230P is exposed.

Referring to FIG. 3H, the mold pattern 230P may be removed, thereby exposing outer wall surfaces of the plurality of lower electrodes LE.

Referring to FIG. 3I, a dielectric film 260 may be formed on the plurality of lower electrodes LE.

The dielectric film 260 may be formed to conformally cover exposed surfaces of the plurality of lower electrodes LE. The dielectric film 260 may be formed to include an aluminum oxide film. The dielectric film 260 may be formed by an ALD process. To form the dielectric film 260, the thin-film forming method described with reference to FIG. 1 or 2, according to the example embodiments of the inventive concept, may be used.

In some embodiments, the dielectric film 260 may include a single film of an aluminum oxide film. In some embodiments, the dielectric film 260 may include a combination of at least one aluminum oxide film and at least one high-k film selected from a tantalum oxide film and a zirconium oxide film (e.g., wherein the two films are stacked on each other).

To form the aluminum oxide film constituting the dielectric film 260 by an ALD process, an aluminum compound according to General Formula (1), for example, an aluminum compound according to Formula (1), may be used as an Al source.

AlCl₃·Et₂S [Formula (1)]

The ALD process for forming the dielectric film 260 may be performed while the temperature of a lower electrode LE is maintained in a range of about 200° C. to about 500° C. For example, the ALD process for forming the dielectric film 260 may be performed while the temperature of the lower electrode LE is maintained in a range of about 200° C. to about 500° C. or a range of about 300° C. to about 500° C.

Referring to FIG. 3J, an upper electrode UE may be formed on the dielectric film 260. The lower electrode LE, the dielectric film 260, and the upper electrode UE, together, may constitute a capacitor 270.

The upper electrode UE may include or be formed of a doped semiconductor, a conductive metal nitride, a metal, a metal silicide, a conductive oxide, or a combination thereof. To form the upper electrode UE, a CVD process, an MOCVD process, a physical vapor deposition (PVD) process, or an ALD process may be used. Additional processes may be subsequently be performed to form a semiconductor device such as a semiconductor chip.

Hereinafter, a specific synthesis example of the aluminum compound, which is included in the thin-film forming composition according to some embodiments, and thin-film forming methods are described. However, the inventive concept is not limited to the following examples.

### Example 1

Synthesis of Aluminum Compound [AlCl₃·Et₂S] of Formula (1)

Aluminum chloride (100 g, 0.75 mol) was dissolved in an n-hexane solution (587 ml, 4.50 mol), followed by introducing diethyl sulfide (161 ml, 1.50 mol) into the solution at 0° C., and then, the components were heated to room temperature and stirred at room temperature for 6 hours. After the reaction was completed, a solvent and a volatile byproduct were removed at reduced pressure, followed by performing vacuum distillation (at 85° C. and 0.169 Torr), thereby obtaining 75.4 g of a compound of Formula (1) (yield 45%).

### (Analysis)

¹H-NMR (solvent: benzene-d6, ppm) δ 0.74 (6H, t, CH₃CH₂SCH₂CH₆), 2.16 (4H, q, CH₃CH₂SCH₂CH₃).

### Evaluation Example

### Evaluation of Properties of Aluminum Compound of Formula (1)

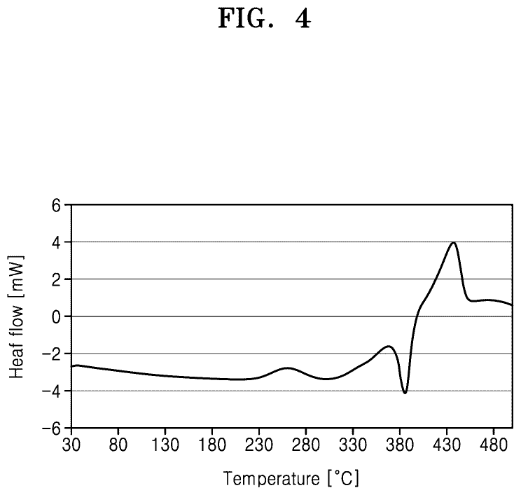

FIG. 4 is a graph illustrating a result of differential scanning calorimetry (DSC) analysis of an aluminum compound according to an embodiment.

As seen from the result of FIG. 4, a pyrolysis peak for the aluminum compound of Formula (1) was not observed up to about 210° C., and it was confirmed that the pyrolysis thereof occurred at about 258° C.

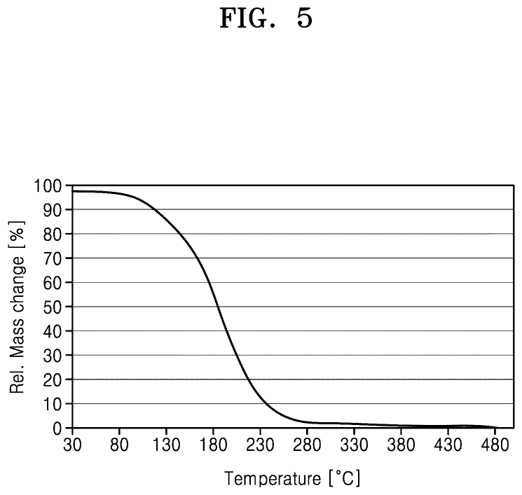

FIG. 5 is a graph illustrating a result of thermogravimetric analysis (TGA) of an aluminum compound according to an embodiment. Regarding FIG. 5, the TGA was performed on 20.8 mg of the aluminum compound of Formula (1) in an argon atmosphere under a heating condition of 10° C./min.

In FIG. 5, the vertical axis represents a relative mass change depending on an increase in temperature. As seen from the result of FIG. 5, the aluminum compound of Formula (1) has rapid vaporization properties, and 95% or more of the aluminum compound of Formula (1) was vaporized at about 280° C. without residue generated due to pyrolysis.

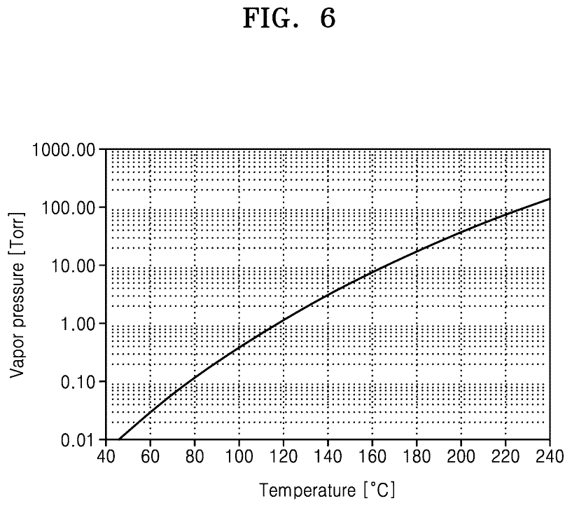

FIG. 6 is a graph illustrating a result of measuring the vapor pressure of an aluminum compound according to an embodiment depending on a change in temperature.

As seen from the result of FIG. 6, it was confirmed that the aluminum compound of Formula (1) has a vapor pressure of about 1 Torr under the condition of 117° C.

### Example 2

### Formation of Aluminum Oxide Film by Using Thin-Film Forming Composition Including Aluminum Compound of Formula (1)

An aluminum oxide film was formed on a silicon substrate (which may be simply referred to as a substrate) by an ALD process by using, as a raw material, the aluminum compound of Formula (1) synthesized in Example 1. Here, ozone gas was used as a reactive gas, and argon was used as a purge gas. The temperature of the substrate was maintained at 200° C. to 500° C. during the formation of the aluminum oxide film.

To form the aluminum oxide film, by taking the following series of process (1) to process (4) as 1 cycle, 70 cycles were repeated.

Process (1): a process in which the vapor of the aluminum compound vaporized at 85° C. corresponding to a heating temperature of a bubbler of the aluminum compound is introduced into a reaction chamber by using argon gas supplied at a flow rate of 25 sccm and adsorbed onto the substrate maintained at 200° C. to 500° C.

Process (2): a process in which the unreacted aluminum compound and byproducts are removed by performing purge for 20 seconds by using argon gas supplied at a flow rate of 3000 sccm.

Process (3): a process in which ozone gas corresponding to the reactive gas is introduced into the reaction chamber at a flow rate of 1500 sccm for 10 seconds and thus reacts with the aluminum compound adsorbed on the substrate.

Process (4): a process in which the unreacted aluminum compound and byproducts are removed by performing purge for 10 seconds by using argon gas supplied at a flow rate of 3000 sccm.

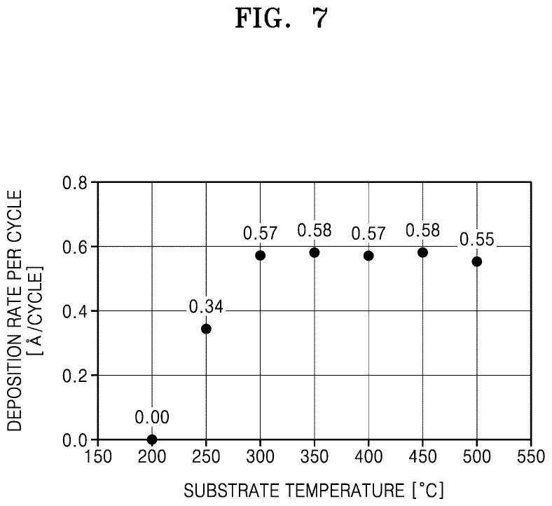

FIG. 7 is a graph illustrating a result of evaluating a deposition rate of an aluminum oxide film, which is formed by using an aluminum compound according to an embodiment as a precursor, depending on the temperature of a substrate.

As seen from the result of FIG. 7, the deposition rate of the aluminum oxide film in a range of 300° C. to 500° C. was about 0.55 Å/cycle to about 0.58 Å/cycle, and the deposition rate of the aluminum oxide film was about 0 Å/cycle at 200° C. and about 0.34 Å/cycle at 250° C. Therefore, it was confirmed that a window range of the ALD process for the aluminum compound of Formula (1) is about 300° C. to about 500° C.

From the above result, it was confirmed that, when the aluminum oxide film is deposited by using the aluminum compound of Formula (1), an ALD behavior having a constant thin-film growth rate was observed, and it was confirmed that a relatively wide ALD window of about 300° C. to about 500° C. may be secured, and that it is easy to adjust the deposition thickness of the aluminum oxide film because the aluminum oxide film has a relatively low deposition rate even at a relatively high process temperature ranging from 300° C. to 500° C. Also, it is easy to evenly and consistently deposit the aluminum oxide film at a constant rate, thereby controlling the thickness despite possible temperature increases and fluctuations, because the rate remains within a relatively small range (e.g., between about 0.55 angstroms per cycle and about 0.58 angstroms per second) for a range of 200° C. (e.g., from about 300° C. to about 500° C.) and remains substantially constant (e.g., between 0.57 and 0.58 angstroms per cycle) for a range of 150° C. (e.g., from about 300° C. to about 450° C.).

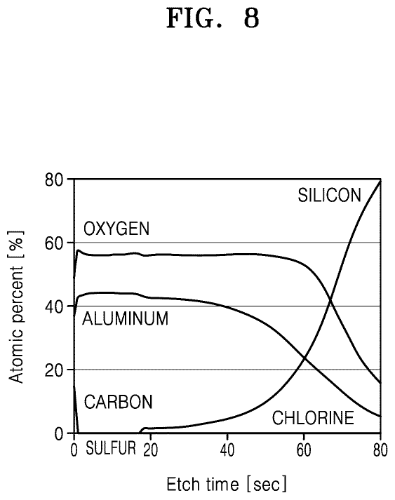

FIG. 8 is a graph illustrating a result of X-ray photoelectron spectroscopy (XPS) depth profiling analysis of an aluminum oxide film, which is formed by using an aluminum compound according to an embodiment as a precursor, for analyzing the concentration composition of the aluminum oxide film. Specifically, FIG. 8 is a graph illustrating a result of XPS depth profiling analysis of an aluminum oxide film, which is formed by using an aluminum compound according to an embodiment as a precursor, when the substrate temperature is 400° C.

As seen from the result of FIG. 8, when the substrate temperature is 400° C., oxygen was detected to be present in an amount of about 57 at % in the obtained aluminum oxide film, and aluminum was detected to be present in an amount of about 43 at % in the obtained aluminum oxide film, whereby it was confirmed that the aluminum oxide film stoichiometrically having a formula of Al₂O₃ was formed. In addition, carbon atoms, chlorine atoms, and sulfur atoms were not detected in the obtained aluminum oxide film, and thus, it was confirmed that the obtained aluminum oxide film does not include impurities generated due to precursor decomposition.

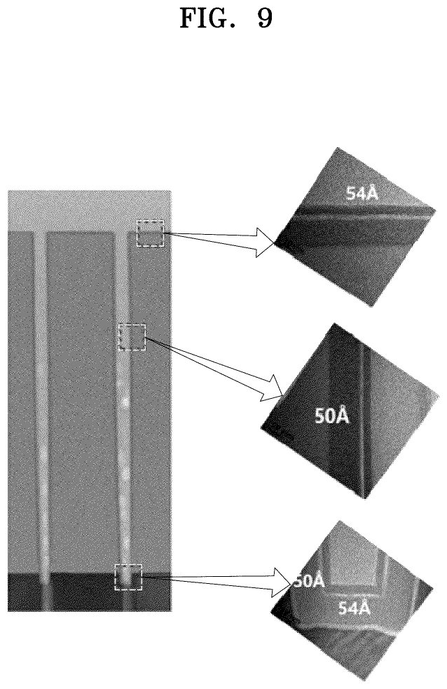

FIG. 9 is a transmission electron microscopy (TEM) image of an aluminum oxide film, which is formed by using an aluminum compound according to an embodiment as a precursor, for analyzing step coverage properties of the aluminum compound. Specifically, FIG. 9 illustrates a TEM image of an aluminum oxide film, which is formed on a pattern having an aspect ratio of about 27:1 by using an aluminum compound according to an embodiment as a precursor, when the substrate temperature is 400° C.

As seen from the result of FIG. 9, when the substrate temperature is 400° C., it was confirmed that the aluminum oxide film formed on a sidewall of the pattern having an aspect ratio of about 27:1 has a thickness of about 50 Å, the aluminum oxide film formed on an upper surface of the pattern has a thickness of about 54 Å, and the aluminum oxide film formed on a bottom surface of a trench between two patterns has a thickness of about 54 Å.

From the above result, when an aluminum oxide film is formed by using the aluminum compound of Formula (1), it was confirmed that the formed aluminum oxide film has a relatively good step coverage of about 92%.

### Comparison Example 1

### Formation of Aluminum Oxide Film by Using Thin-Film Forming Composition Including Trimethylaluminum (TMA)

An aluminum oxide film was formed on a silicon substrate (which may be simply referred to as a substrate) by an ALD process by using TMA as a raw material. Here, ozone gas was used as a reactive gas, and argon was used as a purge gas. The temperature of the substrate was maintained at 200° C. to 500° C. during the formation of the aluminum oxide film.

To form the aluminum oxide film, by taking the following series of process (1) to process (4) as 1 cycle, 60 cycles were repeated.

Process (1): a process in which the vapor of the aluminum compound vaporized at 10° C. corresponding to a heating temperature of a bubbler of the aluminum compound is introduced into a reaction chamber by using argon gas supplied at a flow rate of 25 sccm and adsorbed onto the substrate maintained at 200° C. to 500° C.

Process (2): a process in which the unreacted aluminum compound and byproducts are removed by performing purge for 20 seconds by using argon gas supplied at a flow rate of 3000 sccm.

Process (3): a process in which ozone gas corresponding to the reactive gas is introduced into the reaction chamber at a flow rate of 500 sccm for 10 seconds and thus reacts with the aluminum compound adsorbed on the substrate.

Process (4): a process in which the unreacted aluminum compound and byproducts are removed by performing purge for 10 seconds by using argon gas supplied at a flow rate of 3000 sccm.

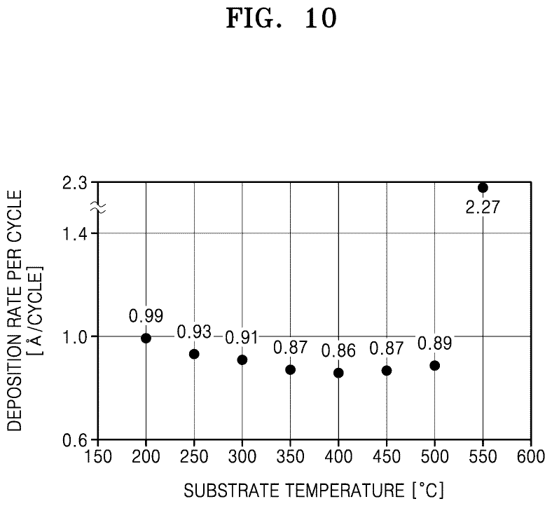

FIG. 10 is a graph illustrating a result of evaluating a deposition rate of an aluminum oxide film, which is formed by using the aluminum compound according to Comparison Example 1 as a precursor, depending on a substrate temperature.

As seen from the result of FIG. 10, it was confirmed that the deposition rate of the aluminum oxide film in a range of 350° C. to 450° C. is about 0.86 Å/cycle to about 0.87 Å/cycle. Therefore, it was confirmed that a window range of the ALD process for TMA is about 350° C. to about 450° C.

From the above result, when the aluminum oxide film is deposited by using TMA, it was confirmed that the ALD process has a relatively narrow ALD process window of about 350° C. to about 450° C. to maintain an even and constant rate of deposition thickness (e.g., compared to a larger process window of about 300° C. to about 450° C., and that it is relatively difficult to adjust the deposition thickness of the aluminum oxide film because the aluminum oxide film has a relatively high deposition rate as compared with depositing an aluminum oxide film by using the aluminum compound of Formula (1).

### Experimental Example 1

### Evaluation of Thermal Stability of Aluminum Compound of Formula (1) and TMA

To evaluate the thermal stability of each of the aluminum compound of Formula (1) and TMA, by taking the following series of process (1) and process (2) as 1 cycle, 500 cycles were repeated. Here, argon was used as a purge gas, and the temperature of a substrate was maintained at 350° C. to 500° C. during process (1) and process (2).

Process (1): a process in which each of the vapor of the aluminum compound vaporized at 85° C. corresponding to a heating temperature of a bubbler of the aluminum compound and the vapor of TMA vaporized at 10° C. corresponding to a heating temperature of a bubbler of the aluminum compound is introduced into a reaction chamber and adsorbed onto the substrate maintained at 350° C. to 500° C.

Process (2): a process in which the unreacted aluminum compound is removed by performing purge for 5 seconds by using argon gas supplied at a flow rate of 3000 sccm.

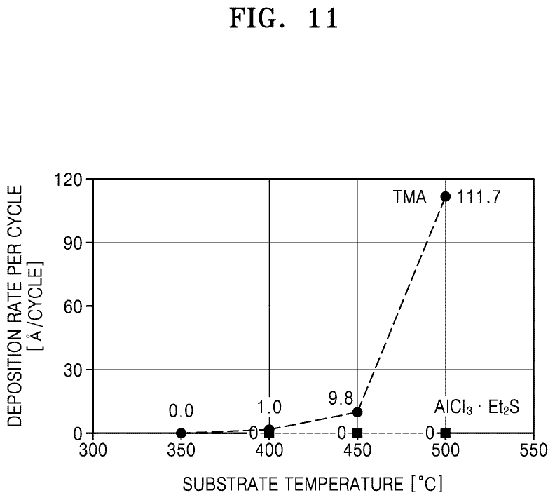

FIG. 11 is a graph illustrating respective results of deposition rates of an aluminum compound according to an embodiment and an aluminum compound according to a comparison example, depending on a substrate temperature.

As seen from the result of FIG. 11, it was confirmed that, while the deposition rate of the aluminum compound of Formula (1) in a range of 350° C. to 500° C. is about 0 Å/cycle, the deposition rate of TMA in the comparison example is about 1.0 Å/cycle at 400° C. and about 111.7 Å/cycle at 500° C.

From the above result, it was confirmed that, while the aluminum compound of Formula (1) is not pyrolyzed at 350° C. to 500° C., TMA is pyrolyzed at a temperature of 400° C. or more. Therefore, it was confirmed that the aluminum compound of Formula (1) has relatively good thermal stability in a range of 350° C. to 500° C. in which an ALD process is performed.

While the inventive concept has been particularly shown and described with reference to embodiments thereof, it will be understood that various changes in form and details may be made therein without departing from the spirit and scope of the following claims.

Terms such as “same,” “equal,” “planar,” “coplanar,” “parallel,” and “perpendicular,” as used herein encompass identicality or near identicality including variations that may occur, for example, due to manufacturing processes. The term “substantially” may be used herein to emphasize this meaning, unless the context or other statements indicate otherwise.

Terms such as “about” or “approximately” may reflect amounts, sizes, orientations, or layouts that vary only in a small relative manner, and/or in a way that does not significantly alter the operation, functionality, or structure of certain elements. For example, a range from “about 0.1 to about 1” may encompass a range such as a 0%-5% deviation around 0.1 and a 0% to 5% deviation around 1, especially if such deviation maintains the same effect as the listed range.

What is claimed is:

1. A thin-film forming composition comprising an aluminum compound represented by General Formula (1):
General Formula (1) 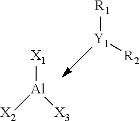
wherein, in General Formula (1), X₁, X₂, and X₃ are each independently a halogen atom, R₁ and R₂ are each independently a C1-C5 alkyl group, and Y₁ is a chalcogen atom.

2. The thin-film forming composition of claim 1, wherein:
R₁ and R₂ are each independently a C1-C3 alkyl group,
X₁, X₂, and X₃ are each independently one selected from the group consisting of Cl, Br, and I, and
Y₁ is one selected from the group consisting of O, S, and Se.

3. The thin-film forming composition of claim 1, wherein the aluminum compound is represented by General Formula (2):
General Formula (2) 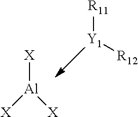
wherein, in General Formula (2), X is a halogen atom, R₁₁ and R₁₂ are each independently a C1-C5 alkyl group, and Y₁ is a chalcogen atom.

4. The thin-film forming composition of claim 1, wherein the aluminum compound has a structure of one selected from the following formulae.

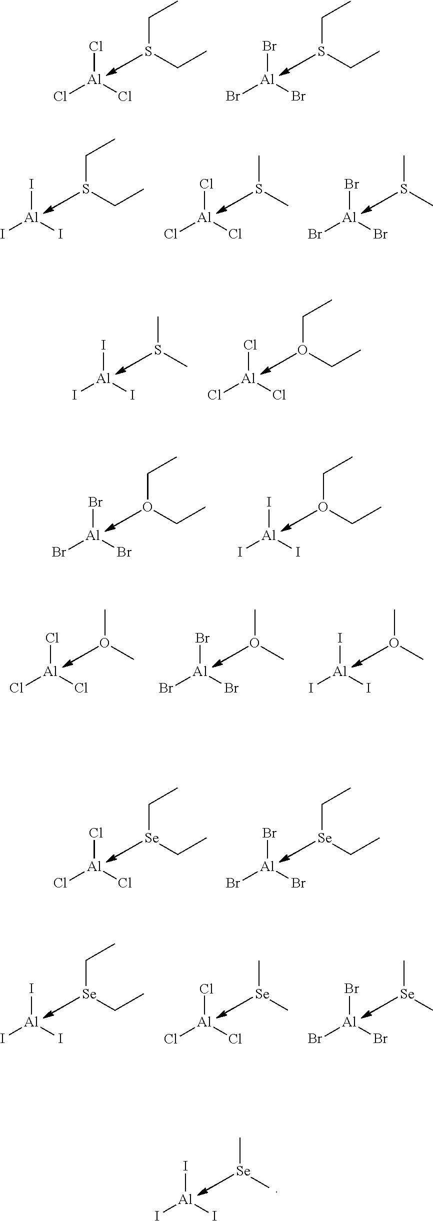

5. A method of forming a thin film, the method comprising:
forming an aluminum-containing film on a substrate by using a thin-film forming composition that comprises an aluminum compound represented by General Formula (1):
General Formula (1) 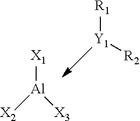
wherein, in General Formula (1), X₁, X₂, and X₃ are each independently a halogen atom, R₁ and R₂ are each independently a C1-C5 alkyl group, and Y₁ is a chalcogen atom.

6. The method of claim 5, wherein:
R₁ and R₂ are each independently a C1-C3 alkyl group,
X₁, X₂, and X₃ are each independently one selected from the group consisting of Cl, Br, and I, and
Y₁ is one selected from the group consisting of O, S, and Se.

7. The method of claim 5, wherein the aluminum compound is represented by General Formula (2):
General Formula (2) 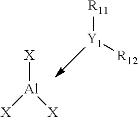
wherein, in General Formula (2), X is a halogen atom, R₁₁ and R₁₂ are each independently a C1-C5 alkyl group, and Y₁ is a chalcogen atom.

8. The method of claim 5, wherein the aluminum compound has a structure of one selected from the following formulae.

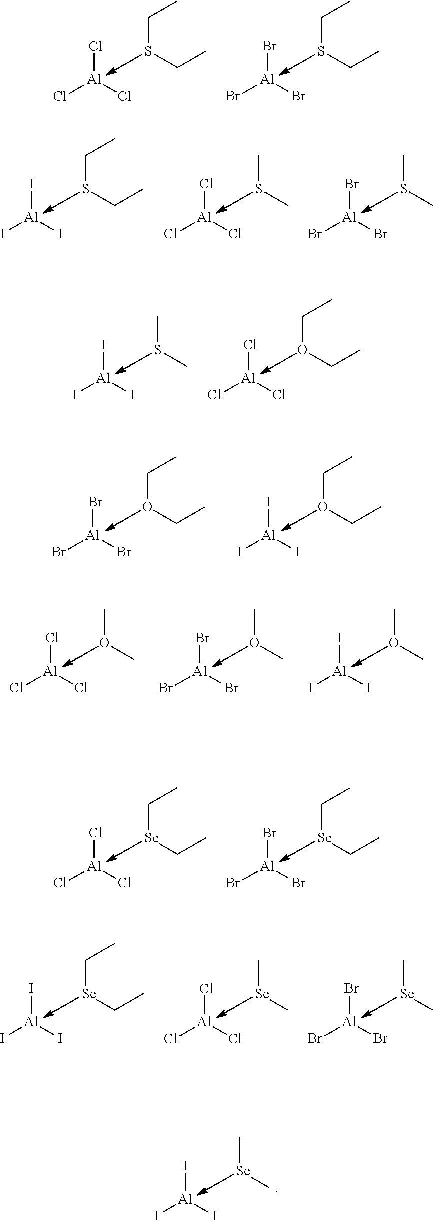

9. The method of claim 5, wherein, in the forming of the aluminum-containing film, a temperature of the substrate is maintained in a range of about 300° C. to about 500° C.

10. The method of claim 5, wherein the forming of the aluminum-containing film comprises:
providing the substrate;
forming an aluminum adsorbed layer by supplying the thin-film forming composition onto the substrate; and
supplying a reactive gas to the aluminum adsorbed layer.

11. The method of claim 10, wherein the reactive gas comprises at least one selected from the group consisting of O₂, O₃, H₂O, H₂O₂, NO, N₂O, NO₂, NH₃, N₂, N₂H₄, H₂, Ar, He, an amine, and a diamine.

12. The method of claim 10, wherein the aluminum-containing film comprises an aluminum oxide film or an aluminum nitride film.

13. The method of claim 10, further comprising supplying a purge gas, before the supplying of the reactive gas after the forming of the aluminum adsorbed layer, and after the supplying of the reactive gas.

14. The method of claim 13, wherein the purge gas comprises one selected from the group consisting of Ar, He, Ne, and N₂.

15. A method of manufacturing a semiconductor device, the method comprising:
forming a lower structure on a substrate; and
forming an aluminum-containing film on the lower structure by using a thin-film forming composition that comprises an aluminum compound represented by General Formula (1):
General Formula (1) 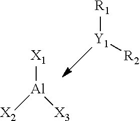
wherein, in General Formula (1), X₁, X₂, and X₃ are each independently a halogen atom, R₁ and R₂ are each independently a C1-C5 alkyl group, and Y₁ is a chalcogen atom.

16. The method of claim 15, wherein the forming of the lower structure comprises forming a lower electrode on the substrate, and
the forming of the aluminum-containing film comprises forming an aluminum oxide film to cover a surface of the lower electrode.

17. The method of claim 16, further comprising forming a dielectric film and an upper electrode in the stated order on the aluminum oxide film.

18. The method of claim 16, wherein, in the forming of the aluminum oxide film, a temperature of the lower electrode is maintained in a range of about 300° C. to about 500° C.

19. The method of claim 15, wherein:
R₁ and R₂ are each independently a C1-C3 alkyl group,
X₁, X₂, and X₃ are each independently one selected from the group consisting of Cl, Br, and I, and
Y₁ is one selected from the group consisting of O, S, and Se.

20. The method of claim 15, wherein the aluminum compound is represented by General Formula (2):
General Formula (2) 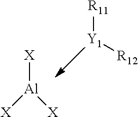
wherein, in General Formula (2), X is a halogen atom, R₁₁ and R₁₂ are each independently a C1-C5 alkyl group, and Y₁ is a chalcogen atom.
